(19) United States
(12) Patent Application Publication
(10) Pub. No.: US 2016/0324495 A1
(43) Pub. Date: Nov. 10, 2016

(54) MOBILE BASE MOUNTED ON AIR CUSHION FOR MEDICAL IMAGING MACHINE

(71) Applicants: Bernard BOUVIER, Buc (FR); Francols KOTIAN, Buc (FR); General Electric Company, Schenectady, NY (US)

(72) Inventors: Bernard BOUVIER, Buc (FR); Francois KOTIAN, Villepreux (FR)

(21) Appl. No.: 15/109,414

(22) PCT Filed: Dec. 31, 2013
(86) PCT No.: PCT/IB2013/002986

§ 371 Date: Jun. 30, 2016

(51) Int. Cl. A61B 6/00

(52) U.S. Cl. CPC A61B 6/4405

(57) ABSTRACT

Exemplary non-limiting embodiment of a mobile base integral with a medical imaging machine or on which a medical imaging machine is to be mounted are described. The mobile base comprises an air cushion platform generating an air cushion between the platform and the ground under the supplied pressurized air so that the platform is then easily movable, the platform being configured to maintain the mobile base and its medical imaging machine stable on the ground when not supplied with pressurized air.

### FIELD OF THE INVENTION

Embodiments of the subject matter disclosed herein generally relates to a mobile base designed to receive a medical imaging system, such as an X-ray machine. This can find particularly advantageous application in the medical imaging and more particularly in the field of medical diagnostic apparatuses.

The mobile base of the invention, when carrying a imaging machine, is particularly designed for a hospital ward, such as a surgical ward, an anesthetic room, a diagnostic unit, an intensive care unit or a ward known as a hybrid ward used to meet the requirements of both angiography rooms and operations rooms.

### DESCRIPTION OF THE RELATED ART

For certain examinations, medical imaging is needed only at the beginning and the end of the operation. Some machines are attached to the ground and cannot be moved away from the patient support.

Mobile base supporting medical imaging system can be found in the prior art. For example, US 2013/0003939 discloses a mobile base with an X-ray machine mounted on it. This mobile base has been a great leap forward in the medical imaging field. The needs of high quality images of the patient and the needs of operating rooms have been fulfilled thanks to a system that is capable of moving the imaging machine.

Embodiments of the mobile base described in US 2013/0003939 include two orientable drive wheels driven respectably by a traction motor and a direction motor. Those motors are coupled to a processing unit and sensors enable to calculate the trajectory and position needed for the operations.

Nevertheless, such a mobile base and an imaging system represent heavy material (several hundred kilograms) but still needs to be moved precisely and stable, especially when the imaging system comprises rotational part, as in US 2013/0003939. The wheels do not provide the imaging system with a perfect stability and the weight of the assembly requires powerful motors to move it.

### BRIEF SUMMARY OF THE INVENTION

An object of the present invention is a mobile base integral with a medical imaging machine or on which a medical imaging machine is to be mounted, characterized in that said base comprises an air cushion platform generating an air cushion between said platform and the ground under the supplying of pressurized air so that said platform is then easily movable , said platform being adapted to maintain the mobile base and its medical imaging machine stable on the ground when not supplied with pressurized air.

Advantageously, the invention is also characterized in that the mobile base further includes at least an orientable drive wheel.

Advantageously, the invention is also characterized in that the orientable wheel is retractable.

Advantageously, the invention is also characterized in that the drive wheel is driven by at least a motor, said motor being coupled to a control unit, which is a configured to input an instruction value on destination, an instructed value on trajectory and data on position of the medical imagining machine, said data on position being provided by at least one sensor, and to generate at output the respective direction and speed for the wheel.

Advantageously, the invention is also characterized in that an apron surrounds the platform, said apron being to come into contact with the ground to limit the air exhaust when the air cushion is formed.

Advantageously, the invention is also characterized in that the mobile base further includes an air re-use system with an air way collecting the air exiting the air cushion.

An object of the invention is also a system comprising a mobile base and a compressor to supply said mobile base with pressurized air.

Advantageously, the invention is also characterized in that said compressor is mounted on said mobile base or on the imaging machine.

Advantageously, the invention is also characterized in that the compressor is independent from the mobile base and from the imaging machine.

An object of the invention is also a medical imaging assembly comprising a medical imaging machine and a base which is integral with the medical imaging machine or on which said medical imaging machine is mounted.

### BRIEF DESCRIPTION OF THE DRAWINGS

The following invention will be understood more clearly from the following description and from the accompanying figures. These figures are given purely by way of an indication and in no way restrict the scope of the invention.

FIG. 1 is a schematic representation of a medical imaging device mounted on a mobile base, according to an embodiment of the invention.

FIG. 2 is a representation of the mobile base according to an embodiment of the invention, when the air cushion is not created.

FIG. 3 is a representation of the mobile base according to an embodiment of the invention, when the air cushion is created.

FIG. 4 is a representation of the mobile base according to another embodiment of the invention, when the air cushion is created.

### DETAILED DESCRIPTION OF THE INVENTION

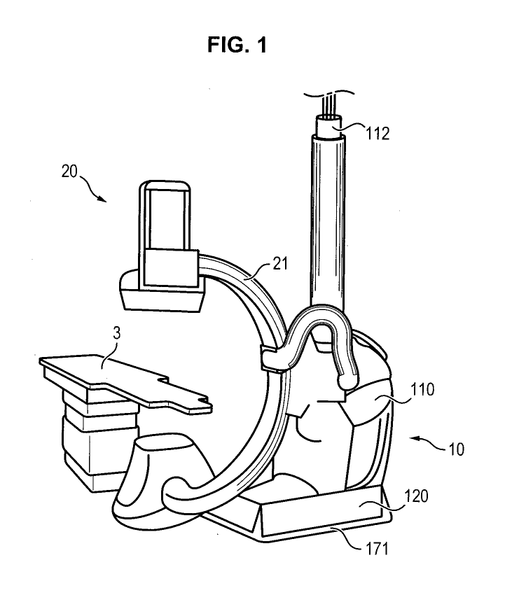

FIG. 1 shows an imaging machine 20 mounted on a mobile base 10 capable of generating an air cushion, in an examination room or surgical ward or hybrid room. The mobile base 10 and the imaging machine 20 will be referred to as assembly 10-20. The room also has an examination table 3, or a bed, on which a patient reclines. In a preferred embodiment of the invention, said medical imaging machine 20 is an X-ray machine as described in the application US 2013/0003939.

During an examination, the imaging machine 20 is shifted in position in working mode so that the organ of the patient, on table 3, to be examined is positioned in the machine range of action.

The mobile base 10 has a support structure 110 which may comprise several parts joined by screwing or by soldering. This structure 110 may also be a cast element. The support structure 110 has a set of structural parts whose joining and geometrical configuration are designed so that: a suitable support on the ground G is provided for the mobile base 10 by a deformation of the set of structural parts forming the support structure 110 and the mobile base 10 is given the rigidity that is necessary and sufficient to eliminate the problem of hyperstatism which may be caused by the structure 110 on the ground G.

The medical imaging machine 20 is connected to a support element 111 of said structure 110, so as to enable all the translations and rotations that could be needed for the imaging machine 20. In another embodiment, said base 10 is integral with the medical imaging machine 20.

The mobile base 10 is also designed so that all its elements balance the weight of the imaging machine 20. The purpose of this balancing is to ensure the stability of the imaging machine 20, even during a shift of a potential moving part 21 of said medical imaging machine 20. The mobile base 10 thus has the role of a counterweight which means that it can maintain the static and dynamic stability of the assembly 10-20. For example, the weight of said assembly 10-20 can reach several hundred kilograms.

The choice of the material, the dimensions, the shape and the thickness of the parts of the structure 110 provides mechanical characteristics of rigidity to the mobile base 10.

### Air Cushion

The mobile base 10 is designed to move the imaging machine 20 on the ground G. To this purpose, said structure 110 of the mobile base 10 comprises a platform 120 which is adapted to generate an air cushion 100 on the ground G. In order to be provided with pressurized air PA to generate the air cushion 100, said mobile base 10 also comprises a pressurized air inlet 121.

In a preferred embodiment, said platform 120 is a plaque comprising said pressurized air inlet 121.

Said plaque 120 comprises a plurality of air tunnel communications 122 on all its area, starting from a pressurized air distributor 130 and ending to the extremity of said plaque 120 facing the ground G, said tunnel communications 122 being preferably straight tunnels. The pressurized air distributor 130 is a device connected to the pressurized air inlet 121 and to said plurality of tunnel communications 122 and is adapted to distribute the pressurized air PA in said communication tunnels 122 according to a certain scheme. This scheme depends on the mass repartition of the assembly 10-20.

When pressurized air PA is provided, the pressurized air PA flows through the pressurized air inlet 121 and is distributed by the pressurized air distributor 130 into the plurality of air tunnel communications 122. Said distributor 130 is adapted to distribute the pressurized air PA into said tunnel communications 122 so as to generate an air cushion 100 between said plaque 120 and the ground G. As aforementioned, to generate a uniform air cushion 100, said distributor 130 distributes the pressurized air PA into said tunnel communications 122 according to the mass repartition of the assembly 10-20.

In order to be as stable as possible, the air cushion 100 is uniform, which means that when pressurized air PA is provided, the assembly 10-20 is undergoing a small vertical translation during the transitional step, creating a gap 101 between the ground G and the plaque 120; and when a steady state is reached, said gap 101 is uniform. Typically, said gap 101 ranges from one millimeter to a centimeter, depending on the pressure of the pressurized air PA. During said steady state, the pressurized air flow through the pressurized air inlet 121 is equal to the air exiting the air cushion 100 through said gap 101.

The pressure and the flow of said pressurized air PA are function of the mass of the assembly 10-20 and the gap 101 between said plaque 10 and the ground G.

The mobile base 10 further includes a regulation system 140 which is controlled manually or by a control unit 150. Said regulation system 140 regulates the pressurized air inlet 121 and/or the distributor 130 to control the gap 101 created by the air cushion 100. In order to improve the uniformity of said air cushion 100 and/or to control said gap 101, the mobile base 10 includes, according to a certain embodiment, sensors 141 adapted to measure the horizontality of the mobile base 10 or the width of the gap 101.

### Compressor

The pressurized air PA is provided to the distributor 130 by a compressor 160. Said compressor 160 is connected to said pressurized air inlet 121 through a pressurized air connection 161 and receives air from an air supplier.

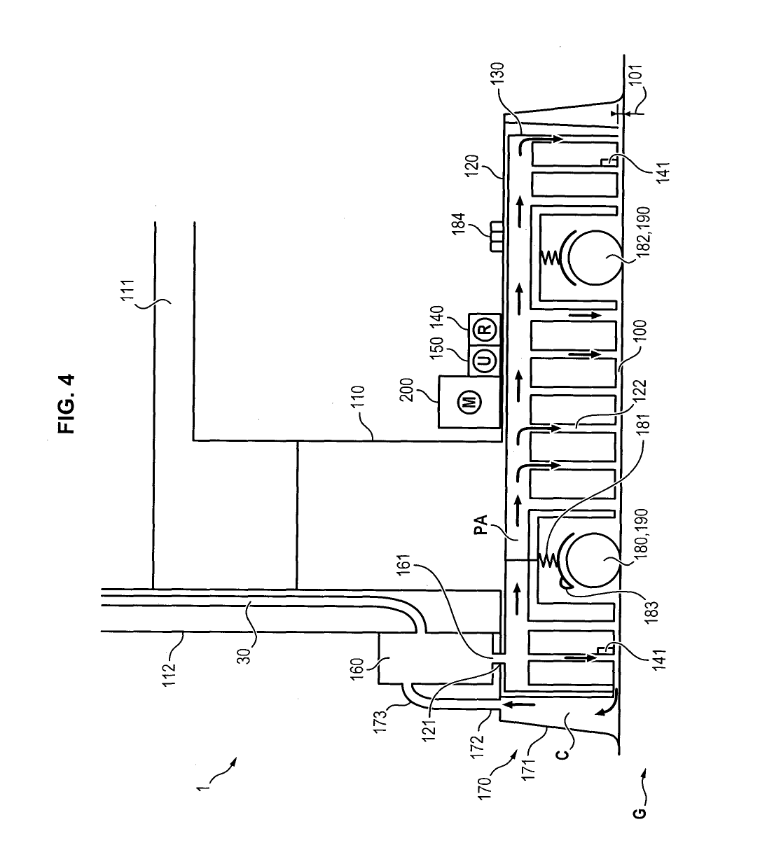

In one embodiment, said compressor 160 is mounted directly on the mobile base 10. The air supplier is then preferably a flexible tube 30 (see FIG. 4).

In a preferred embodiment, said mobile base 10 further includes a tube collector 112 which is a structure adapted to gather the wires and tubes whose one extremity is connected to the assembly 10-20. Said tube collector 112 is typically a hollow rigid tube with one extremity almost reaching the ceiling of the room, and the tube collector 112 contains in particular the flexible tube 30 of the air supplier, the pressurized air connection 161, electrical wires to supply the assembly 10-20 with energy, and data wires. Such a tube collector 112 optimizes the ergonomics of the assembly 10-20.

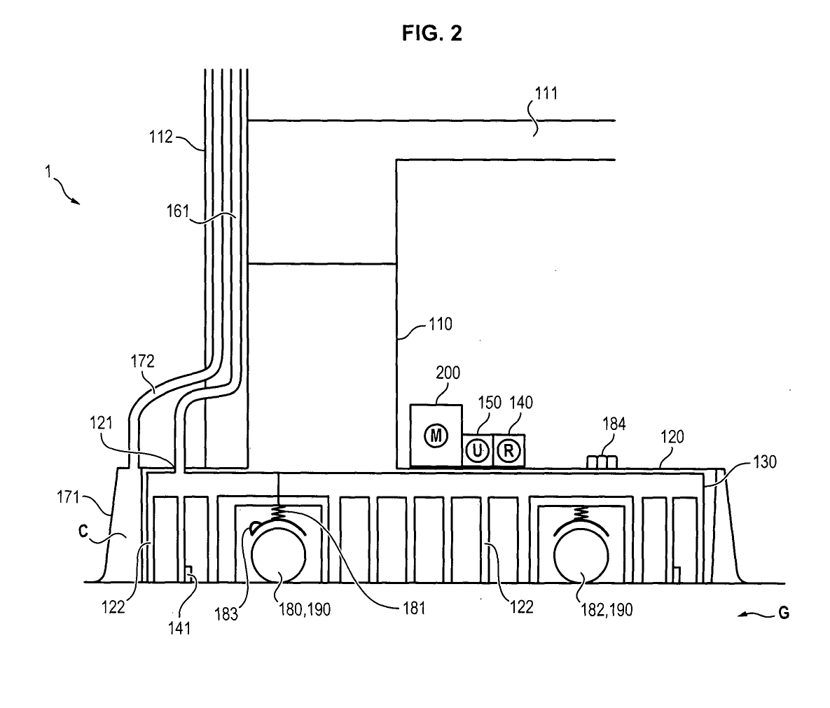

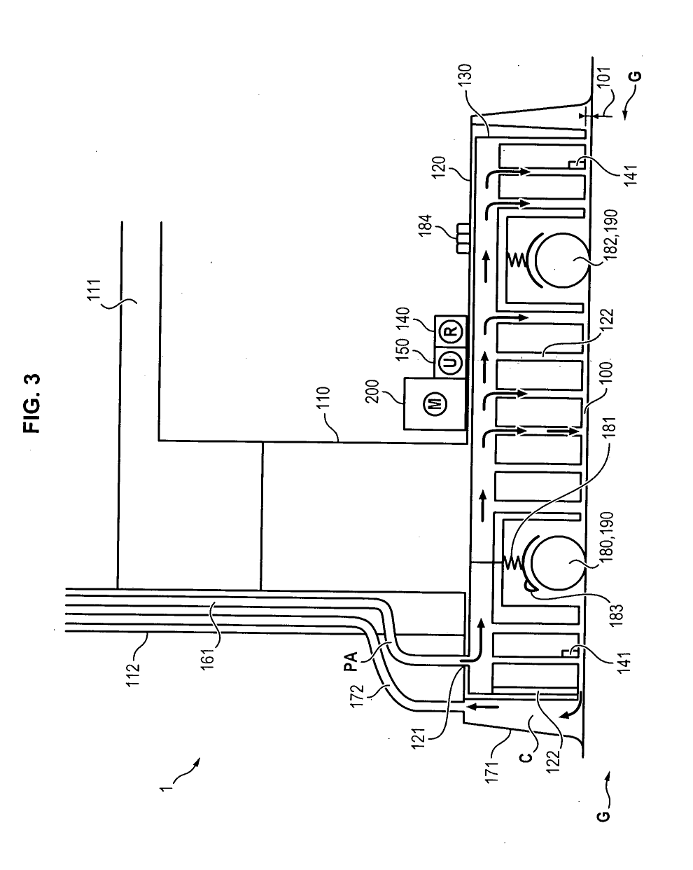

In a preferred embodiment, said compressor 160 is independent from the mobile base 10 and can be located in the examination room or outside. The pressurized air connection 161 is preferably a second flexible tube which is also constrained into said tube collector 112. Having the compressor 160 independent reduces the mass and the potential noise of the assembly 10-20 and improves its ergonomics (see FIG. 2-3).

Said compressor 160 delivers a flow and a pressure adapted to create the air cushion 100.

### Recycling Unit

In another embodiment, the mobile base 10 includes also an apron 171. Said apron 171 surrounds the platform 120 and comes into contact with the ground G. The apron 171 is made of a flexible material adapted to maintain contact with said ground G when the mobile base 10 is moving and pressurized air PA is provided for the air cushion 100. Said apron 171 prevents air exhaust from the mobile base 10 into the room.

Along with said apron 171, in another embodiment, the mobile base 10 further includes a recycling unit 170, said recycling unit 170 being adapted to reuse the air exiting the air cushion 100. The recycling unit 170 comprises said apron 171, a used air way 172 and an air output 173, said used air way 172 connecting a cavity C under the apron 171 to the air output 173. Said cavity C is formed by the apron 171, the platform 120 and the ground G.

The apron 171 gathers the used air exiting from the air cushion 100 and flowing into said cavity C, said used air being then directed towards the used air way 172. The air output 173 is either connected to the air supplier (embodiment not shown on figures) which provides said compressor 160 with air or directly connected to the compressor 160 (see FIG. 4). Thus, it is described a close loop of the air entering the compressor 60, limiting any kind of contamination or degradation of the conditions of the room.

Typically, the used air way 172 is made of a third flexible tube collecting with one extremity the used air from the cavity C and connecting with the other extremity, through said air output 173, to the air supplier 30 or the compressor 60.

In the preferred embodiment wherein the compressor 160 is independent from the mobile base 10, said third flexible tube 172 is directed towards the tube collector 112, still in order to optimize the ergonomics of the assembly 10-20.

### Wheels & Motorization

The mobile base 10 further includes at least an orientable drive wheel 180. The platform 120 of the structure 110 is adapted to receive the wheel 180 so that said wheel 180 can be fully inserted inside said platform 120. Thus the platform 120 can lie on the ground G when pressurized air PA is not applied. Besides, the wheel G is mounted on a spring 181 or a hydraulic cylinder adapted to maintain contact between the wheel 180 and the ground G when the air cushion 100 is created.

Said wheel 180 is in a preferred embodiment a four direction orientable wheel 180 adapted to move the assembly 10-20 in every direction. In another embodiment, two orientable drive wheels 180, 182 are included. Said wheel 180 rotates at a speed A and is oriented at an angle alpha and the wheel 182 rotates at the speed B and is oriented at an angle beta. The speeds A and B are often different and the angles alpha and beta are often different. These different speeds and angles of the two orientable drive wheels 180, 182 enable the imaging machine to be moved in an examination room in minimizing the volume traversed by said apparatus to the maximum extent.

More details about said wheels 180, 182 can be found in US 2013/0003939.

The mobile base 10 furthermore has a freewheel system 190, capable of undergoing rotational movements induced by the orientable drive wheel(s) 180, 182. Said freewheel system 190 facilitates the movements of the assembly 10-20. Said orientable drive wheel(s) 180, 182 can also be declutched to become said freewheel system 190 (see FIG. 2-4).

Besides, a breaking device 183 is provided, preferably on said free wheel system 190, and enables the immobilization of the assembly 10-20 even when the air cushion 100 is created. This breaking device 183 can be controlled manually or remotely for example by means of said control unit 150.

More details about said breaking device 183 can be found in US 2013/0003939.

The orientable drive wheel 180 is driven by at least one motor 200 mounted on the mobile base 10. Thanks to the air cushion 100 which support most of the mass of the assembly 10-20, the needed torque to move the assembly 10-20 is considerably decreased compared to prior art described in US 2013/0003939 and frictions between the ground G and the wheel(s) 180, 182 are sufficient to enable the movement of the assembly 10-20. Consequently, the size of the motor 200 is reduced compared to prior art and enables to gain weight.

Said motor 200 is coupled to the control unit 150 which is configured to output the respective angle and speed for the wheel(s) 180, 182. Purposely, at least one sensor (not shown on figures) can be included on said mobile base 10 and/or the imaging machine 20 and/or in the examination room. Said sensor provides the control unit 150 with the position of the medical imaging machine 20. The instruction values on destination and trajectory, coupled to the data on position are provided to the control unit 150, through communication buses or wirelessly. Such a control unit 150 and sensors, along with programs, man-machine interface, routines, Cartesian representation, etc., are described in the application US 2013/0003939 and can be fully applied to the present invention.

The mobile base further comprises means 184 to manually handle the assembly 10-20.

### Description of a Functioning System

When pressurized air PA is not provided, the platform 120 of the structure 110 of the mobile base is directly on the ground G, giving the assembly 10-20 an excellent stability. Each wheel 180, 182 is located inside said mobile platform 120, the spring 181 or hydraulic cylinder being compressed.

This configuration is adapted to enable the processing of the imaging machine 20, and is particularly adapted for said imaging machine 20 comprising rotational part 21. As aforementioned, the issue of hyperstatism is overcome with the material of said structure 110, and especially of said platform 120. The stability is then improved compared to prior art.

When it is needed to move the imaging machine 20, the pressurized air inlet 121 opens and the pressurized air PA flows through the distributor 130 and the communication tubes 122 to reach the ground G. Under the pressure of said pressurized air PA, the platform 120 will undergo a vertical force which triggers the vertical translation of the assembly 10-20. The small gap 101 between said platform 120 and the ground G is created. The wheels 180, 181 maintain contact with ground since the spring 181 or the hydraulic cylinder can extend. In one embodiment, the control unit 150, according to the instruction it has been given and the information coming from the sensors controls the angle and the speed of the wheels 180, 181 to move the assembly 10-20 thanks to the motor 150. In another embodiment, an operator can move the assembly 10-20 by said means 184 to manually handle the assembly 10-20.

Once the desired position is reached, the pressurized air inlet 121 closes. The platform 120 of the structure 110 comes into contact with the ground G and the spring 181 or hydraulic cylinder get compressed so that the wheels 180, 182 can be fully located inside said platform 120.

1. A mobile base integral with a medical imaging machine or on which a medical imaging machine is to be mounted, comprising:
an air cushion platform configured to generate an air cushion between the platform and the ground when pressurized air is supplied so that movability of the platform is improved relative to the movability of the platform when pressurized air is not supplied, the platform being further configured to maintain the mobile base and its medical imaging machine stable on the ground when not supplied with pressurized air.

2. The mobile base according to claim 1, further comprising at least one orientable drive wheel.

3. The mobile base according to claim 2, wherein the at least one orientable wheel is retractable.

4. The mobile base according to claim 2, wherein the at least one orientable drive wheel is driven by at least one motor, the at least one motor coupled to a controller configured to input at least an instruction value on destination, an instructed value on trajectory and data on position of the medical imagining machine, the data on position being provided by at least one sensor, and to generate and output the respective direction and speed for the wheel.

5. The mobile base according to claim 1, further comprising an apron that surrounds the platform, the apron configured to come into contact with the ground to limit air exhaust when the air cushion is formed.

6. The mobile base according to claim 5, further comprising an air re-use system with an air way collecting the air exiting the air cushion.

7. A medical imaging system comprising:
a medical imaging machine;
a mobile base comprising an air cushion platform configured to generate an air cushion between the platform and the ground when pressurized air is supplied so that movability of the platform is improved relative to the movability of the platform when pressurized air is not supplied, the platform being further configured to maintain the mobile base and its medical imaging machine stable on the ground when not supplied with pressurized air, wherein the mobile base is integral with the medical imaging machine or configured to support the medical imaging machine when mounted thereon; and
a compressor configured to supply the mobile base with pressurized air.

8. The system according to claim 7, wherein the compressor is mounted on the mobile base or on the imaging machine.

9. The system according to claim 7, wherein the compressor is independent from the mobile base and from the imaging machine.

10. A medical imaging assembly comprising:
a medical imaging machine; and
a base comprising an air cushion platform configured to generate an air cushion between the platform and the ground when pressurized air is supplied so that movability of the platform is improved relative to the movability of the platform when pressurized air is not supplied, the platform being further configured to maintain the mobile base and its medical imaging machine stable on the ground when not supplied with pressurized air,
wherein the mobile base is integral with the medical imaging machine or configured to support the medical imaging machine when mounted thereon.

11. The medical imaging assembly according to claim 10, further comprising a compressor configured to supply pressurized air to the mobile base.

12. The mobile base according to claim 1, further comprising a compressor configured to supply pressurized air to the mobile base.
